(19) United States
(12) Patent Application Publication
(10) Pub. No.: US 2026/0166213 A1
(43) Pub. Date: Jun. 18, 2026

(54) METHOD AND APPARATUS FOR SUCTION DURING SURGICAL PROCEDURES

(71) Applicant: E8, Inc., Los Gatos, CA (US)

(72) Inventors: Peter DAVIS, Dana Point, CA (US); Joshua Allen DAVISON, Buffalo, NY (US); Michael Thomas SZYMCZAK, Batavia, NY (US)

(73) Assignee: E8, Inc., Los Gatos, CA (US)

(21) Appl. No.: 19/532,283

(22) Filed: Feb. 6, 2026

Related U.S. Application Data
(63) Continuation of application No. PCT/US2024/041804, filed on Aug. 9, 2024.
(60) Provisional application No. 63/518,508, filed on Aug. 9, 2023.
(60) Provisional application No. 63/596,929, filed on Nov. 7, 2023.

(51) Int. Cl. A61M 1/00

(52) U.S. Cl. CPC A61M 1/87; A61M 1/7411; A61M 1/86; A61M 2205/0266; A61M 2205/273

(57) ABSTRACT

Disclosed herein are methods and devices for providing suction at a target area during a medical procedure. A medical device can be provided and can comprise a handle and an elongated shaft extending from the handle. The elongated shaft can comprise a longitudinal axis, a proximal end, a distal end, and a central lumen extending to a distal tip having an opening. The medical device can comprise a plurality of cutting elements within the central lumen, the plurality of cutting elements extending from an inner surface of the elongated shaft, the plurality of cutting elements being angled with respect to the longitudinal axis such that an edge of each of the plurality of cutting elements adjacent to the proximal end is tapered, the plurality of cutting elements being spaced apart both along the longitudinal axis and circumferentially offset with respect to an inner circumference cross-section of the elongated shaft.

### RELATED APPLICATION INFORMATION

This application is a continuation of PCT Application No. PCT/US2024/041804, filed Aug. 9, 2024 which claims priority to U.S. Provisional Application No. 63/518,508, filed on Aug. 9, 2023, and U.S. Provisional Application No. 63/596,929, filed on Nov. 7, 2023, the entireties of which are incorporated by reference.

### TECHNICAL FIELD

The devices and methods disclosed herein relate to surgical suction devices having the ability to reduce clogging during surgery.

### BACKGROUND OF THE INVENTION

Surgical aspirators, commonly referred to as suction wands, sucker tips, or sucker devices, allow a physician to remove fluids, tissue, and/or other debris from a surgical site in or on a patient. Surgical aspirators can include a tip and/or shaft inserted into a surgical site, wound, or other bodily site. The shaft is generally elongated in shape and can include a handle or grip section for holding the aspirator as well as for coupling a vacuum source to the device. Such vacuum sources can be a tube fluidly connected to a suction pump that provides suction at the tip.

Aspiration or suction during surgery is typically necessary to clear the surgical field from tissue debris and unwanted blood that could lead to (or is already causing) intracerebral hemorrhage, deep vein thrombosis, stroke, embolism, etc. In most cases, conventional devices clog frequently, which requires removal of the sucker device from the surgical site. In such cases, the sucker device is either exchanged or cleared using another device or wire. In either case, blockage of the sucker devices during a procedure results in longer procedure times, increased costs due to the use of additional devices, and/or risk of harm to a patient where the procedure is time sensitive.

Therefore, there exists a need for a suction device that efficiently captures and removes unwanted material from a target area.

### SUMMARY OF THE INVENTION

Disclosed herein is a medical device for providing suction at from a suction source a target area during a medical procedure. The medical device can comprise a handle and an elongated shaft extending from the handle. The elongated shaft can comprise a longitudinal axis, a proximal end, a distal end, and a central lumen extending to a distal tip having an opening. The medical device can comprise a plurality of cutting elements within the central lumen, the plurality of cutting elements extending from an inner surface of the elongated shaft, the plurality of cutting elements being angled with respect to the longitudinal axis such that an edge of each of the plurality of cutting elements adjacent to the proximal end is tapered, the plurality of cutting elements being spaced apart both along the longitudinal axis and circumferentially offset with respect to an inner circumference cross-section of the elongated shaft.

When the suction source pulls a material through the opening, the material can engage the plurality of cutting elements such that the material is cut incrementally while being suctioned towards the proximal end of the elongated shaft.

At least two of the plurality of cutting elements can be circumferentially spaced 90 degrees from each other. The plurality of cutting elements can be circumferentially spaced over at least 360 degrees. Adjacent cutting elements along the longitudinal axis can be longitudinally spaced apart at variable distances along the elongated shaft. The elongated shaft can be rigid. The plurality of cutting elements can be configured to flex when engaged with the material. The plurality of cutting elements can comprise elastic materials. The plurality of cutting elements can be made of nitinol. The handle can be removable from the elongated shaft. The handle can be disposable.

The plurality of cutting elements can comprise a plurality of rows of tines extending longitudinally along the elongated shaft. The plurality of rows of tines can be spaced apart 90 degrees circumferentially from each other along the elongated shaft. The plurality of cutting elements can each comprise a length of more than half of a diameter of the elongated shaft. Points or edges of each of the plurality of cutting elements can overlap radially.

The handle can further comprise a suction opening configuration to control suction from the suction source. The handle can further comprise a rotatable barb that connects to a suction tube. The suction tube can couple the suction source and the central lumen of the elongated shaft. The rotatable barb and the suction tube can be connected via a rotatable suction connector. An irrigation tube can be positioned concentrically outside of the central lumen.

In some variations, a method for providing suction from a suction source at a target area during a medical procedure is disclosed. The method can comprise introducing a medical device to the target area. The medical device can comprise a handle and an elongated shaft extending from the handle. The elongated shaft can comprise a longitudinal axis, a proximal end, a distal end, and a central lumen extending to a distal tip having an opening. The medical device can comprise a plurality of cutting elements within the central lumen, the plurality of cutting elements extending from an inner surface of the elongated shaft, the plurality of cutting elements being angled with respect to the longitudinal axis such that an edge of each of the plurality of cutting elements adjacent to the proximal end is tapered, the plurality of cutting elements being spaced apart both along the longitudinal axis and circumferentially with respect to an inner circumference of the elongated shaft. When the suction source pulls a material through the opening, the material can engage plurality of cutting elements such that the material is cut incrementally while being suctioned towards the proximal end of the elongated shaft.

In some variations, a system for capturing and cutting material at a target area during a medical procedure. The system can comprise a handle and an elongated shaft extending from the handle. The elongated shaft can comprise a longitudinal axis, a proximal end, a distal end, and a central lumen. The system can comprise a plurality of cutting elements within the central lumen, the plurality of cutting elements extending from an inner surface of the elongated shaft. The system can comprise a distal tip coupled to the distal end of the elongated shaft. The distal tip can comprise an aspiration window. The system can comprise a suction source configured for applying suction to pull material through the aspiration window and into the central lumen. The system can comprise an umbrella portion at the distal end of the elongated shaft. The umbrella portion can be slidable axially to engage with material pulled through the aspiration window.

When the suction source pulls a material through the opening, the material engages plurality of cutting elements such that the material is cut incrementally while being suctioned towards the proximal end of the elongated shaft. The plurality of cutting elements can be angled with respect to the longitudinal axis such that an edge of each of the plurality of cutting elements adjacent to the proximal end is tapered. The plurality of cutting elements can be curved tines spaced apart circumferentially with respect to an inner circumference of the elongated shaft.

### BRIEF DESCRIPTION OF THE DRAWINGS

FIGS. 1A and 1B illustrate respective perspective and side views of the medical suction device in accordance with one variation of the invention.

FIG. 2 illustrates a variation of a distal end of the shaft for use with the suction device.

FIGS. 3A and 3B illustrate partial cross-sectional views of another variation of a shaft comprising a plurality of stationary cutting elements in the shaft of the suction device.

FIGS. 4A to 4E illustrate additional cut tube configurations where portions of the wall are folded into a lumen of the tube to form cutting elements.

FIGS. 5A and 5B illustrate a variation of a shaft having an opening in the distal that has a specific geometry to match a cutting element.

FIG. 5C illustrates another variation of a shaft that can comprise a beveled tip or taper at a distal end of the shaft.

FIG. 6 illustrates a system comprising an elongated shaft and a removable, disposable handle.

FIG. 7 illustrates a suction device according to one variation of the present invention.

FIGS. 8A and 8B illustrate side views of a variation of another variation of a suction device.

FIGS. 9A and 9B illustrate respective perspective and side views of another variation of a suction device.

### DETAILED DESCRIPTION

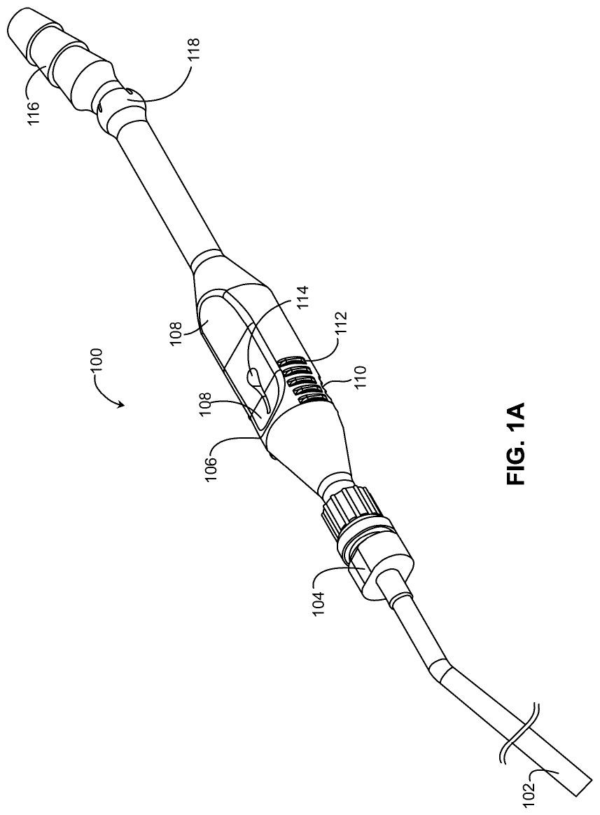

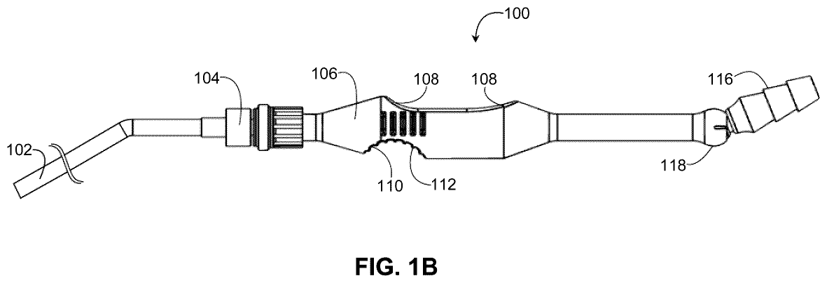

FIGS. 1A and 1B illustrate respective perspective and side views of the medical suction device 100 in accordance with one variation of the invention. The device 100 can comprise an elongated shaft 102 having a central lumen within. The elongated shaft 102 can be made of a cylindrical structure fabricated from metal, polymeric, composite material, or a combination thereof. The device 100 can be introduced to the desired target area via convention methods.

The suction device 100 can include a rotational luer fitting 104 with a cammed surface that allows for rotation of the shaft 102 relative to a handle 106. The suction device 100 can comprise any number of curved surfaces 108 and finger rests 110 to assist in the manipulation of the device and to improve the ergonomic features of the suction device 100. The surfaces 108, finger rests 110, and/or other surfaces of the handle 106 can include any number of ridges 112 to assist in gripping the suction device 100.

The suction device 100 can further comprise a control window 114 configured to accommodate a control to manipulate suction from a suction source (not shown). The suction device 100 can comprise a barb connector 116 coupling with a suction tubing.

The suction device 100 can comprise a ball-socket fitting 118 to accommodate the barb connector 116 having a ball surface to permit articulation of the barb connector 116 relative to the handle 106 of the suction device 100. The ball-socket fitting 118 can comprise an O-ring to reduce leakage during the procedure. The ball-socket fitting 118 can allow for rotational movement of about 10 degrees to about 20 degrees (e.g., about 15 degrees).

In other variations, the ball-socket fitting 118 can be angled such that the suction tube is substantially perpendicular to the longitudinal axis of the handle 106, allowing for rotation of about 180 degrees.

The shaft 102 can be angled for improved visualization through the distal end of the shaft 102. As will be described below, the shaft 102 can comprise a plurality of cutting elements configured to assist in keeping the shaft 102 free from blockages.

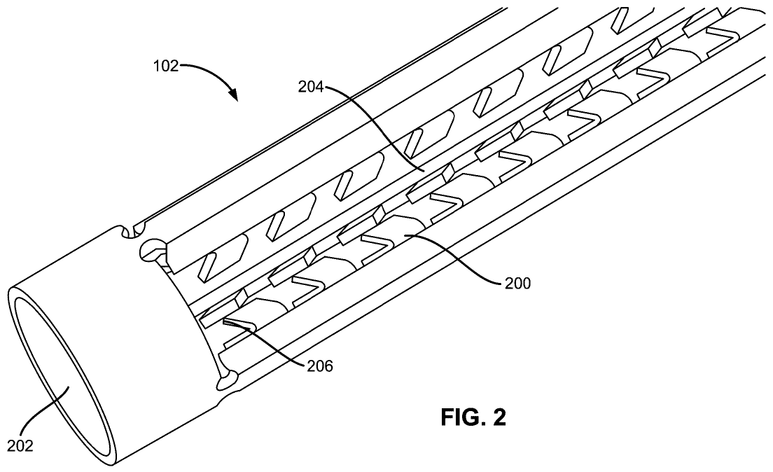

FIG. 2 illustrates a variation of a distal end of the shaft 102 for use with the suction device 100. In this variation, portions of the shaft wall can be folded inwards to form a plurality of cutting elements 200. The shaft 102 can be laser cut such that portions of the wall are then folded toward the center of the lumen. The shaft 102 can comprise an outer layer or sleeve (e.g., a heat shrunk jacket) to allow vacuum to be drawn within the lumen. The shaft can comprise an opening 202 at a distal end through which debris or material can enter the central lumen 204 when the distal end is advanced to a target area.

The plurality of cutting elements 200 can be within the central lumen 204 and can extend from an inner surface of the elongated shaft 102. The plurality of cutting elements 200 can be angled with respect to the longitudinal axis such that a edge 206 of each of the one more cutting elements 200 is directed towards the distal end of the elongated shaft 102. In other variations, the cutting elements 200 can extend substantially perpendicular to the longitudinal axis of the shaft 102 such that the edge 206 extends in a normal direction with respect to the longitudinal axis. The edge 206 of each of the cutting elements 200 adjacent to the proximal end of the shaft 102 can be tapered.

The plurality of cutting elements 200 can be spaced apart longitudinally along the longitudinal axis. In some variations, the plurality of cutting elements 200 can be spaced apart at regular intervals along the shaft 102. In some variations, the plurality of cutting elements 200 can be spaced apart at variable intervals along the shaft 102.

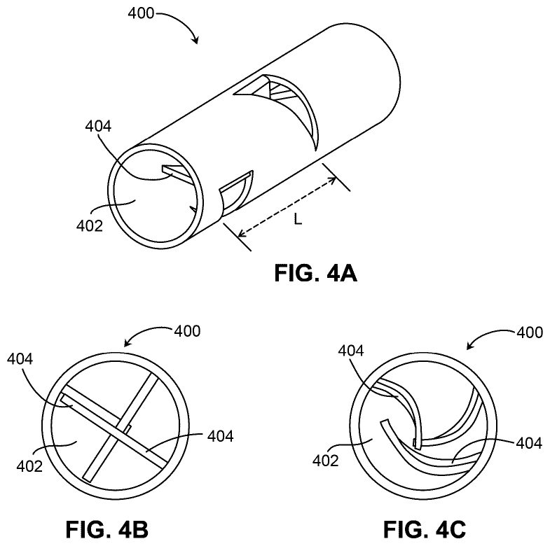

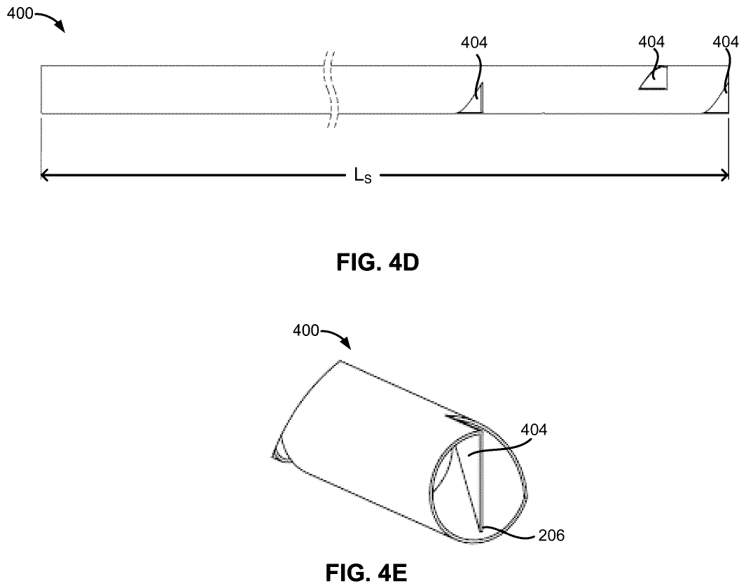

The plurality of cutting elements 200 can be circumferentially offset with respect to an inner circumference of the elongated shaft 102. As seen below in FIG. 4B for example, adjacent cutting elements 200 can be offset from each other by 90 degrees (i.e., a first cutting element extends from the inner surface of the shaft 102 at an angle of 90 degrees from a second cutting element). Accordingly, material that is suctioned through the central lumen can be incrementally cut as the suction moves the material proximally. This configuration can allow the cutting elements 200 to cut the material until the material is small enough to be suctioned through an exit port on the handle (not shown).

The plurality of cutting elements 200 can comprise an elastic material (e.g., nitinol). This allows the cutting elements 200 to flex when engaged with any material passing through. Accordingly, the combination of the suction energy through the central lumen and the material itself engaging with the cutting elements 200 can result in a vibratory force that assists in cutting the material. The shaft 102 can be made of a rigid material to accommodate for flexing of the cutting elements 200. The outer diameter of the shaft 102 can be heat shrunk to accommodate for the suction applied through the central lumen 204.

In some variations, the plurality of cutting elements 200 can comprise a plurality of rows of cutting elements extending longitudinally along the elongated shaft 102. The plurality of rows of cutting elements 200 can be spaced apart 90 degrees circumferentially from each other along the elongated shaft 102. In some variations, the plurality of rows of cutting elements 200 can be spaced apart at varying angles circumferentially from each other along the elongated shaft 102.

In some variations, the shaft 102 can function as an insert for an outer tube or shaft of the suction device 100, which provides a closed surface to draw vacuum within the insert.

In some variations, the cutting elements 200 can be produced by forming two halves of a tube with internal structures that can be welded or conjoined to form an airtight tube capable of aspiration.

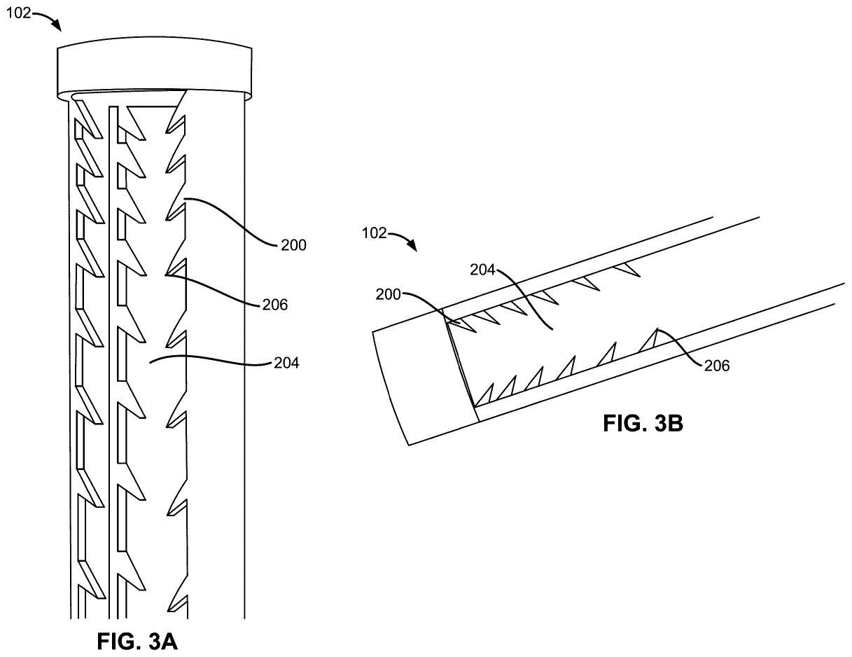

FIGS. 3A and 3B illustrate partial cross-sectional views of another variation of a shaft 102 comprising a plurality of stationary cutting elements 200, blades, or tines in the shaft 102 of the suction device 100. As shown in FIG. 3A, the cutting elements 200 can be positioned in rows longitudinally and at an angle such that an edge of the cutting elements 200 adjacent to the proximal end is tapered towards a proximal end of the device. The rows of cutting elements 200 can be spaced apart circumferentially along the inner surface of the shaft 102 (e.g., spaced apart about 90 degrees from each other), although varying spacing can also be used.

In some variations, the cutting elements 200 can be positioned in a spiral direction and/or other geometries such that they can cut or change the shape of materials that could clog the suction device 100. In some variations, cutting elements 200 can be oriented in a proximal or distal direction, can extend short, long, or variable distances into the lumen of the shaft 102. The cutting elements 200 can also be non-symmetric and/or can have a variable pitch. The number of cutting elements 200 and can be located at any initial distance from the distal end of the shaft 102.

In some variations, the shaft 102 can function as an insert for an outer tube of the suction device 100, which provides a closed surface to draw vacuum within the insert.

FIG. 4A illustrates one variation of a shaft 400 where portions of the wall are folded into a lumen 402 of the shaft 400 to form axially spaced cutting elements 404. In the cross-section illustrated in FIG. 4B, the cutting elements 404 can be relatively straight and can extend across a length of more than half of a diameter of the shaft 400 but not entirely across the lumen 402. In this configuration, adjacent cutting elements 404 can be offset from each other by about 90 degrees (i.e., a first cutting element extends from the inner surface of the shaft 400 at an angle of 90 degrees from a second cutting element). The cutting elements 404 can be spaced apart axially along the shaft 400 at a length L. As seen in FIG. 4B, points or edges 206 of each of the plurality of cutting elements 404 can overlap radially. The variation shown in FIG. 4B illustrates the cutting elements 404 as being circumferentially spaced over 360 degrees of the interior of the lumen 402.

In the variation illustrated in FIG. 4C, the cutting elements 404 can be folded within the lumen of the shaft 400 in an arcuate shape. In this variation, the cutting elements 404 can extend partially across the diameter of the lumen 402. Such cutting elements can be curved tines that are axially spaced along the elongated shaft 400 and circumferentially offset with respect to a cross-section of the elongated shaft. In this variation, there are three cutting elements 404 circumferentially spaced over 180 degrees of the lumen 402. It should be appreciated that variations of the device can include any number of cutting elements spaced over a full or partial circumference of the device.

FIGS. 4D and 4E illustrate yet another variation of shaft 400 comprising cutting elements 404 axially spaced at varying intervals over the length Lₛ of the shaft. Length Lₛ can be about 10 cm to about 20 cm (e.g., 14.5 cm). Cutting elements 404 can comprise straight edges, curved edges, or a combination thereof. As seen in FIG. 4E the most distal cutting element 404 can be part of the distal tip of the shaft 400. In this variation, the cutting element 404 fold opens up in distalmost surface area which can engage a larger clot than just a circular end.

In some variations, the cutting elements 404 can be oriented straight in a radial direction. In other variations, the cutting elements 404 can be oriented curved in a radial direction.

It should be understood that any variation of the disclosed cutting elements can be used in any variation of the shaft. It should also be understood that any variation of different orientations and geometries of the disclosed cutting elements can be used together within a variation of the shaft.

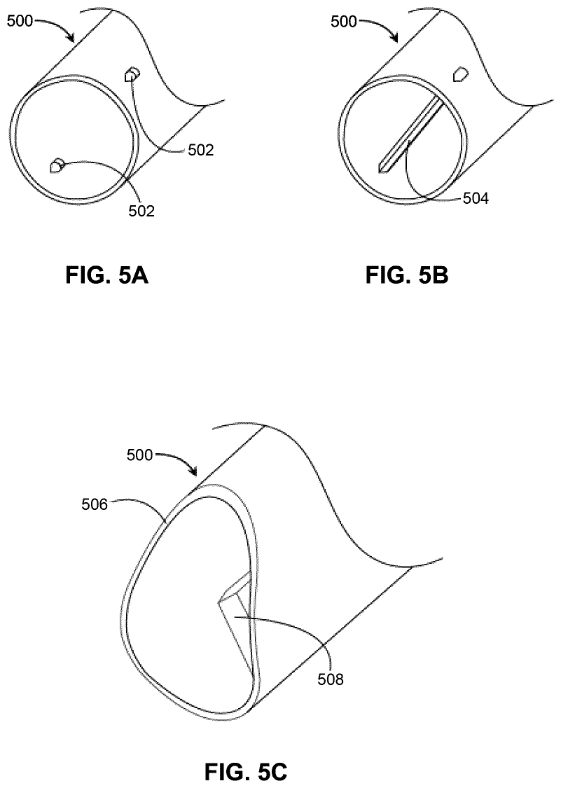

FIGS. 5A and 5B illustrate a variation of a shaft 500 having an opening 502 in the distal that has a specific geometry to match a cutting element 504. Accordingly, the cutting element 504 can have a cross-sectional profile that matches the geometry of the opening 502. For coupling of the cutting element 504 to the opening 502, the cutting element 504 can be glued or welded into the opening 502. In other variations, the shaft 500 can include multiple cutting elements 504 for multiple openings.

FIG. 5C illustrates another variation of a shaft 500 that can comprise a beveled tip 506 or taper at a distal end of the shaft 500. The beveled tip 506 can increase the surface area of the opening 502 for clot or material engagement, allowing the suction device to capture additional material.

In some variations, a static internal cutting element 508 can be used within the central lumen of the shaft 500. In some variations, the shaft 500 can include markers and/or bands that allow for tracking the travel of the distal tip of the catheter using X-ray or another non-invasive imaging modality. In some variations, the marker bands can be made from radiopaque metal or other trackable material and therefore laser cut to enable the formation of the static cutting element 508 once folded inward, into the shaft lumen. Therefore, when a clot is pulled into the shaft it travels through the cutting element, thus macerating the clot into smaller pieces for easier and faster aspiration.

In some variations, multiple static cutting elements 508 can be laser cut into the shaft 500 such that after folding the blades into the lumen the folded portion can form a helical pattern that can generate an impeller function, thus re-sizing the tissue or clot for faster aspiration.

In some variations, multiple static cutting elements 508 can be folded inward and have a geometry such that a guidewire can still pass through central lumen and the blades can remain close to the internal wall of the hypotube.

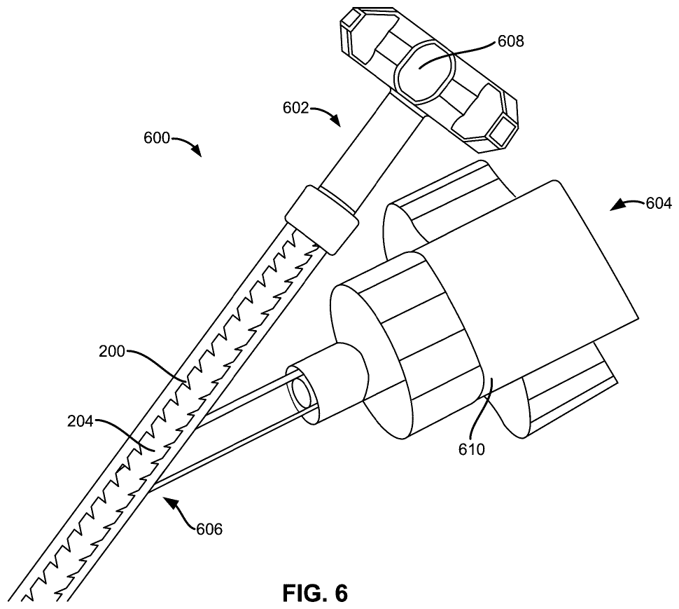

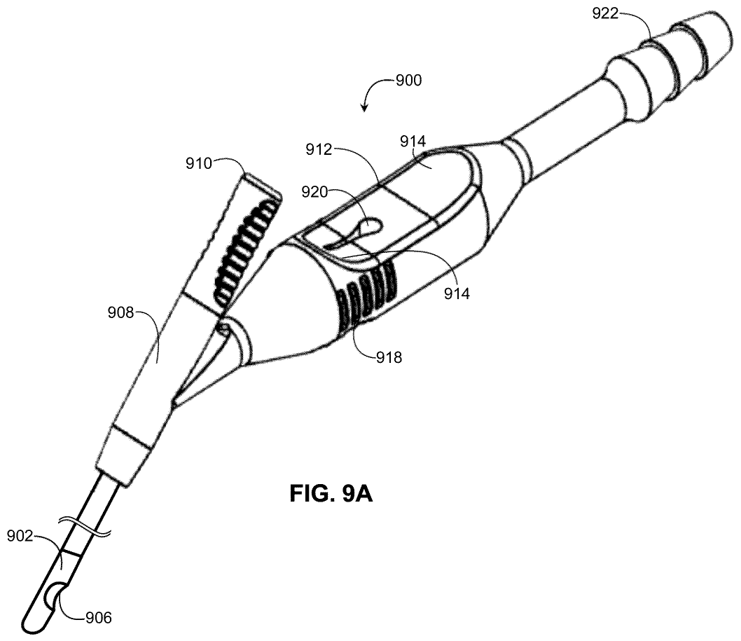

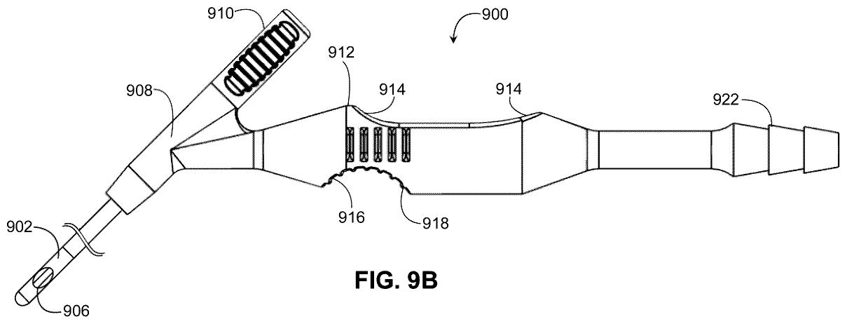

FIG. 6 illustrates a system 600 comprising an elongated shaft 602 and a removable, disposable handle 604. The elongated shaft 602 can be coupled to the handle 604 via an opening on the handle (not shown in FIG. 6, see example in FIG. 9A) at a Y-connection. This allows the handle 604 to be removable from the elongated shaft 602 and disposable as desired. The shaft 602 can comprise a rotational handle 608 which allows rotational or sliding motion of the shaft 602 relative to the handle when coupled via the Y-connection.

The suction source can provide suction to the central lumen 204 to pull material proximally for cutting via cutting elements as described previously herein. Material pulled proximally can be accordingly removed via an exit port on the handle 604 via suction towards a proximal end of the handle 604.

The handle 604 can comprise a rotatable suction connector 610 that can couple to a suction tubing leading to a suction source. Rotating the suction connector 610 can assist the user in coupling the suction tubing from a barb connector (not shown) on within the handle 604.

In this variation, the plurality of cutting elements 200 can taper towards the proximal end of the shaft 602 while the points or edges of the cutting elements 200 are extend perpendicular to the longitudinal axis of the shaft 602. In other variations, points or edges of the cutting elements 200 can extend towards a distal end of the shaft 602.

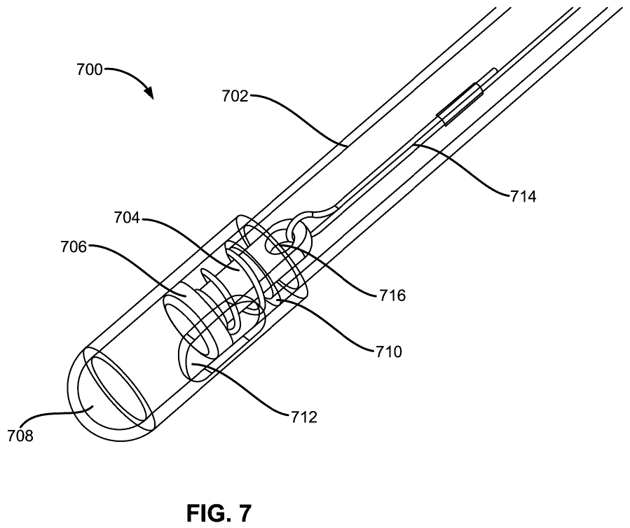

FIG. 7 illustrates a suction device 700 according to one variation of the present invention. The suction device 700 can comprise a shaft 702 and an umbrella portion 704 extending from a distal end of the shaft 702. The umbrella portion 704 can have an umbrella head 706 shaped as a flat cap, a domed cap, or a similar structure. The umbrella portion 704 can cover the entirety of the circumferential area of the atraumatic tip 708.

The umbrella portion 704 can be coupled to an elastic member 710 which can be compressed and relaxed such that the umbrella portion 704 is slidable proximally and distally. After suction of material into the suction device 700, the umbrella portion 704 can be pulled proximally to pull material into the lumen of the shaft 702. The suction device 700 can comprise transition proximally to a larger shaft where the material can be aspirated or reach an exit port where the trapped material may be removed through an exit port or opening (not shown). The umbrella portion 704 can reach the distal end of the device 700 towards an atraumatic tip 708 positioned at the distal end of the shaft 702.

The atraumatic tip 708 can comprise an aspiration window 712 on a sidewall thereof. The aspiration window 712 can comprise an oval shape, a circular shape, or any other shape for material to pass through. The aspiration window 712 can be positioned at the target area of the anatomy such that material can be sucked into the suction device 700 and into a lumen of the shaft 702.

The umbrella portion 704 can be coupled to a handle (not shown) with an actuator via a mechanical connection such as a wire 714 or similar structure. The actuator (e.g., a trigger, ratchet, joystick, etc.) can permit movement of the umbrella portion 704 proximally to pull material away from the aspiration window 712. The wire 714 can be coupled to the umbrella portion 704 which comprises an opening 716 for the wire 714 to be threaded into.

In some variations, the umbrella portion 704 can be pulled proximally to be engaged with a cutting mechanism (i.e., the cutting elements described above) to disrupt or cut the material into smaller pieces capable of being aspirated.

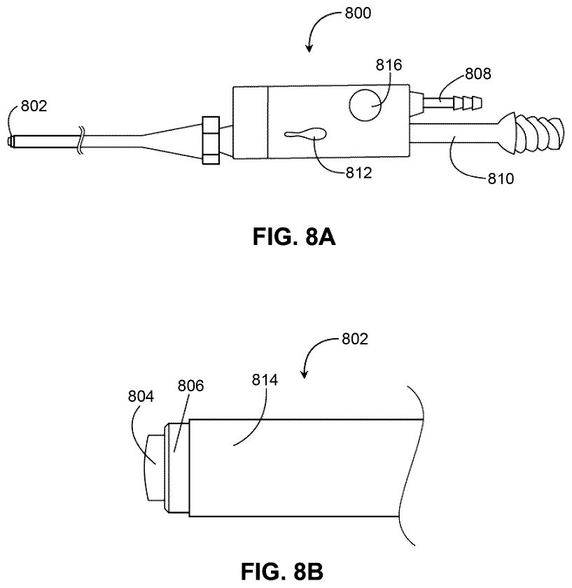

FIGS. 8A and 8B illustrate another variation of a suction device 800 comprising a distal tip 802 that includes a suction tube 804 that can comprise a plurality of cutting elements as discussed above. The suction tube 804 can be surrounded by an external irrigation tube 806 which is concentrically positioned around the suction tube 804. The irrigation tube 806 can be configured to deliver irrigation to the target area.

The suction device 800 can further comprise a vacuum fitting 808 and a fluid fitting 810. The vacuum fitting 808 can be in fluid communication with a suction tube 804 and can include barbs for attaching to an external suction tube coupled to a suction source (not shown). The fluid fitting 810 can be in fluid communication with an irrigation tube 806 and can include barbs for attaching to an external irrigation tube coupled to an irrigation source (not shown). The suction device 800 can further comprise one or more control windows 812 that allow the user to control the suction and/or irrigation of the device 800.

In some variations, a gap between the suction tube 804 and the irrigation tube 806 can allow for bipolar energy delivery to the distal end of the device 800 as the suction tube 804 and the irrigation tube 806 are coupled to an energy source (not shown) and can function as bi-polar electrodes. In such a case, the device 800 can include an external insulating layer 814. Accordingly, the device can further include a control switch 816 for controlling the application of RF energy.

FIGS. 9A and 9B illustrate respective perspective and side views of another variation of the suction device 900. As shown, the suction device 900 can comprise a shaft 902 that can include a plurality of cutting elements 904 (as described above) configured to assist in keeping the shaft 902 free from blockages. In this variation, the suction device 900 can comprise a window 906 at the tip of the shaft 902.

The suction device 900 can be designed to seat the shaft 902 at a shaft portion 908 such that a shaft handle 910 allows for rotation of the shaft relative to a device handle 910 while still allowing vacuum to be applied at the window 906. The suction device 900 can further comprise one or more control windows 920 that allow the user to control the suction and/or irrigation of the device 900.

The shaft handle 910 and the device handle 912 can comprise any features such as curved surfaces, ribs, etc., to improve handling of the suction device 900. The suction device 900 can include any number of curved surfaces 914 and finger rests 916 to assist in the manipulation of the device and to improve ergonomic features of the device. The surfaces 914, finger rests 916, and/or other surfaces of the handle 912 can include any number of ridges 918 to assist in gripping the suction device 900.

Additionally, the suction device 900 can include an integral barb connector 922 for coupling to a vacuum source.

It is noted that any feature of a particular variation described above can be combined into alternate variations of the devices. As for other details of the present invention, materials and manufacturing techniques may be employed as within the level of those with skill in the relevant art. The same may hold true with respect to method-based aspects of the invention in terms of additional acts that are commonly or logically employed. In addition, though the invention has been described in reference to several examples, optionally incorporating various features, the invention is not to be limited to that which is described or indicated as contemplated with respect to each variation of the invention.

Various changes may be made to the invention described and equivalents (whether recited herein or not included for the sake of some brevity) may be substituted without departing from the true spirit and scope of the invention. Also, any optional feature of the inventive variations may be set forth and claimed independently, or in combination with any a plurality of of the features described herein. Accordingly, the invention contemplates combinations of various aspects of the embodiments or combinations of the embodiments themselves, where possible. Reference to a singular item, includes the possibility that there are plural of the same items present. More specifically, as used herein and in the appended claims, the singular forms “a,” “and,” “said,” and “the” include plural references unless the context clearly dictates otherwise.

It is important to note that where possible, aspects of the various described embodiments, or the embodiments themselves can be combined. Where such combinations are intended to be within the scope of this disclosure.

1. A medical device for providing suction from a suction source at a target area during a medical procedure, the medical device comprising:
a handle;
an elongated shaft extending from the handle, the elongated shaft comprising a longitudinal axis, a proximal end, a distal end, and a central lumen extending to a distal tip having an opening;
a plurality of cutting elements within the central lumen, the plurality of cutting elements extending from an inner surface of the elongated shaft, the plurality of cutting elements being angled with respect to the longitudinal axis such that an edge of each of the plurality of cutting elements adjacent to the proximal end is tapered, the plurality of cutting elements being spaced apart both along the longitudinal axis and circumferentially with respect to an inner circumference of the elongated shaft; and
wherein when the suction source pulls a material through the opening, the material engages the plurality of cutting elements such that the material is cut incrementally while being suctioned towards the proximal end of the elongated shaft.

2. The medical device of claim 1, wherein at least two of the plurality of cutting elements are circumferentially spaced 90 degrees from each other.

3. The medical device of claim 1, wherein the plurality of cutting elements circumferentially spaced over at least 360 degrees.

4. The medical device of claim 1, wherein adjacent cutting elements along the longitudinal axis are longitudinally spaced apart at variable distances along the elongated shaft.

5. The medical device of claim 1, wherein the elongated shaft is rigid.

6. The medical device of claim 1, wherein the plurality of cutting elements are configured to flex when engaged with the material.

7. The medical device of claim 1, wherein the plurality of cutting elements comprise elastic materials.

8. The medical device of claim 5, wherein the plurality of cutting elements are made of nitinol.

9. The medical device of claim 1, wherein the handle is removable from the elongated shaft, wherein the handle is disposable.

10. The medical device of claim 1, wherein the plurality of cutting elements comprise a plurality of rows of tines extending longitudinally along the elongated shaft.

11. The medical device of claim 10, wherein the plurality of rows of tines are spaced apart 90 degrees circumferentially from each other along the elongated shaft.

12. The medical device of claim 1, wherein the plurality of cutting elements each comprise a length of more than half of a diameter of the elongated shaft.

13. The medical device of claim 12, wherein points or edges of each of the plurality of cutting elements overlap radially.

14. The medical device of claim 1, wherein the handle further comprises a suction opening configured to control suction from the suction source.

15. The medical device of claim 1, wherein the handle further comprises a rotatable barb that connects to a suction tube, the suction tube coupling the suction source and the central lumen of the elongated shaft.

16. The medical device of claim 15, wherein the rotatable barb and the suction tube are connected via a rotatable suction connector.

17. The medical device of claim 1, further comprising an irrigation tube positioned concentrically outside of the central lumen.

18. A method for providing suction from a suction source at a target area during a medical procedure, the method comprising:
introducing a medical device to the target area, the medical device comprising:
a handle;
an elongated shaft extending from the handle, the elongated shaft comprising a longitudinal axis, a proximal end, a distal end, and a central lumen extending to a distal tip having an opening;
a plurality of cutting elements within the central lumen, the plurality of cutting elements extending from an inner surface of the elongated shaft, the plurality of cutting elements being angled with respect to the longitudinal axis such that an edge of each of the plurality of cutting elements adjacent to the proximal end is tapered, the plurality of cutting elements being spaced apart both along the longitudinal axis and circumferentially with respect to an inner circumference of the elongated shaft; and
wherein when the suction source pulls a material through the opening, the material engages the plurality of cutting elements such that the material is cut incrementally while being suctioned towards the proximal end of the elongated shaft.

19. A system for capturing and cutting material at a target area during a medical procedure, the system comprising:
a handle;
an elongated shaft extending from the handle, the elongated shaft comprising a longitudinal axis, a proximal end, a distal end, and a central lumen;
a plurality of cutting elements within the central lumen, the plurality of cutting elements extending from an inner surface of the elongated shaft;
a distal tip coupled to the distal end of the elongated shaft, wherein the distal tip comprises an aspiration window;
a suction source configured for applying suction to pull material through the aspiration window and into the central lumen;
an umbrella portion at the distal end of the elongated shaft, wherein the umbrella portion is slidable axially to engage with material pulled through the aspiration window; and
wherein when the suction source pulls a material through the aspiration window, the material engages the plurality of cutting elements such that the material is cut incrementally while being suctioned towards the proximal end of the elongated shaft.

20. The system of claim 19, wherein the plurality of cutting elements are angled with respect to the longitudinal axis such that an edge of each of the plurality of cutting elements adjacent to the proximal end is tapered.

21. The system of claim 19, wherein the plurality of cutting elements are curved tines spaced apart circumferentially with respect to an inner circumference of the elongated shaft.
